(19) United States
(12) Patent Application Publication
(10) Pub. No.: US 2002/0198235 A1
(43) Pub. Date: Dec. 26, 2002

(54) Use of benzothiopenes to treat and prevent prostate cancer

(75) Inventor: David B. Agus, Beverly Hills, CA (US)

Correspondence Address: Pillsbury Winthrop LLP, Intellectual Property Group, Suite 2800, 725 South Figueroa Street, Los Angeles, CA, 90017-5406, US

(73) Assignee: CEDARS-SINAI MEDICAL CENTER

(21) Appl. No.: 10/142,087

(22) Filed: May 9, 2002

Related U.S. Application Data
(60) Provisional application No. 60/290,307, filed on May 10, 2001.

(51) Int. Cl. A61K 31/4535

(52) U.S. Cl. 514/320

(57) ABSTRACT

Disclosed herein is a method for treating and preventing prostate cancer, the method comprising administering to a mammal a benzothiopene having the formula

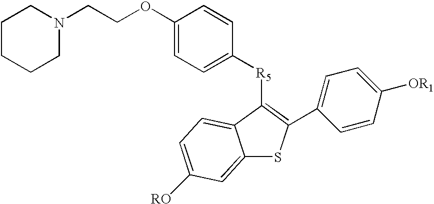

or pharmaceutically acceptable salts thereof, wherein R and R₁ are each independently selected from the group consisting of hydrogen, —COR₂, —COR₃, and R₄; R₂ is selected from the group consisting of hydrogen, C1-C14 alkyl, C1-C3 chloroalkyl, C1-C3 fluoroalkyl, C5-C7 cycloalkyl, C1-C4 alkoxy, and phenyl; R₃ is phenyl with at least one substitution selected from the group consisting of C1-C4 alkyl, C1-C4 alkoxy, hydroxy, nitro, chloro, fluoro, trichloromethyl, and trifluoromethyl; R₄ is selected from the group consisting of C1-C4 alkyl, C5-C7 cycloalkyl, and benzyl; and R₅ is selected from the group consisting of oxygen and —C═O.

This application claims the benefit of priority under 35 U.S.C. §119(e) of U.S. Provisional Application Serial No. 60/290,307 filed May 10, 2001, the contents of which are hereby incorporated by reference.

### FIELD OF THE INVENTION

Embodiments of the present invention are directed to methods for treating and preventing prostate cancer, in particular androgen-independent prostate cancer, by administering to a patient a benzothiopene, such as raloxifene or arzoxifene.

### BACKGROUND OF THE INVENTION

Several types of cancer can affect the prostate gland, including adenocarcinoma, sarcoma, squamous cell carcinoma, and ductal transitional carcinoma. Adenocarcinoma is the most common of these and is one of the most common malignancies in men over 50 years old. In the United States, over 200,000 men become diagnosed with this type of cancer every year.

Most cancers of the prostate gland have androgen receptors. They depend on testosterone and other androgens for their growth. Hormone ablative therapy—therapy which chemically interferes with androgens or the organs that produce it—is therefore standard in the treatment of prostate cancer. Such therapy can prolong survival of men with prostate cancer from 3 to 5 years or more.

Chemical hormone ablative therapy includes the administration of drugs that block the hormonal pathways that lead to androgen synthesis. In the male, the hypothalamus secretes gonadotropin releasing hormone (“GnRH”), which stimulates the pituitary gland to secrete luteinizing hormone (“LH”); LH, in turn, stimulates the testes to produce testosterone. Synthetic analogs of GnRH and LH are therefore administered to reduce levels of androgens circulating within the body. These analogs compete with their natural counterparts for binding to GnrH receptors, thereby down-regulating the receptors and interrupting the testosterone production cycle. Estrogen also blocks androgen production, but can compromise cardiovascular health and so is rarely used. Antiandrogen drugs, such as flutamide, bicalutamide, or nilutamide, are also available. These bind to androgen receptors on prostate cancer cells, preventing their activation, and thereby limiting the action of natural androgens.

Surgical hormonal ablative therapy comprises surgical removal of one or both testes (orchiectomy), the testosterone-producing organ in the male. One may increase the effectiveness of this treatment still further by combining it with anti-androgens.

Androgen-independent prostate cancer (also called hormone refractory prostate cancer) does not depend on androgens for its growth; as a result, hormone ablative therapy has little effect on it. Even therapies that are highly effective at treating androgen-dependent cancers have been shown to be ineffective when applied to patients with androgen-independent cancer.

Androgen-independent cancer is difficult to treat. One can decrease the size of prostate inflammation associated with the cancer, such as with corticosteroids and other anti-inflammatory agents, but such treatment has no effect on the cancer itself. For this reason, a basic medical text still teaches that “There is no standard therapy for hormone refractory prostate cancer.” M. H. Beers and R. Berkow, eds., Merck Manual of Diagnosis and Therapy, 1658 (1999). According to this text, cytotoxic and biologic agents “are being investigated,” but “their superiority to corticosteroids alone has not been proved.”

There is therefore a significant need in the art for a satisfactory treatment of androgen-independent prostate cancer. Such a treatment could have a dramatic impact on the health of older men, among whom prostate cancer is common.

### SUMMARY OF THE INVENTION

It is an object of the present invention to provide a therapy for the treatment of prostate cancer, and in particular for the treatment of prostate cancer that responds poorly to hormone ablative therapy or does not respond to such therapy at all. In other words, it is an object of the present invention to provide a last hope, a therapy for the treatment of prostate cancer in those cases where patients have had—until now—little hope for long-term survival.

Disclosed herein is a method of treating prostate cancer that is surprisingly effective at treating androgen-independent prostate cancer, dramatically reversing the progression of the disease in patients previously diagnosed as having only months to live. The method comprises administering to patients an effective amount of a compound having the formula

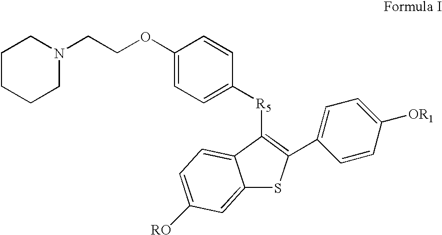

or pharmaceutically acceptable salts thereof. In a preferred embodiment, the compound is raloxifene, the compound of Formula I wherein R and R₁ are both hydrogen and R₅ is —C═O. In another preferred embodiment, the compound is arzoxifene, the compound of Formula I wherein R and R₁ are both hydrogen and R₅ is oxygen.

### DETAILED DESCRIPTION OF THE INVENTION

The method of the invention comprises administering to a patient an effective amount of a compound having the formula

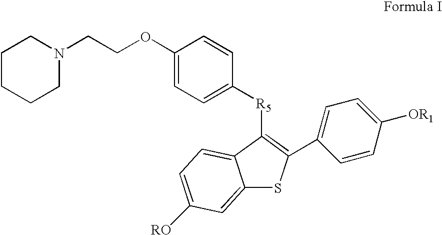

or pharmaceutically acceptable salts thereof, wherein R and R₁ are each independently selected from the group consisting of hydrogen, —COR₂,—COR₃, and R₄; R₂ is selected from the group consisting of hydrogen, C1-C14 alkyl, C1-C3 chloroalkyl, C1-C3 fluoroalkyl, C5-C7 cycloalkyl, C1-C4 alkoxy, and phenyl; R₃ is phenyl with at least one substitution selected from the group consisting of C1-C4 alkyl, C1-C4 alkoxy, hydroxy, nitro, chloro, fluoro, trichloromethyl, and trifluoromethyl; R₄ is selected from the group consisting of C1-C4 alkyl, C5-C7 cycloalkyl, and benzyl; and R₅ is selected from the group consisting of oxygen and —C═O. Such compounds, as well as methods of preparing them, are described in detail in U.S. Pat. No. 4,418,068 (“the '068 patent”), the disclosure of which is hereby incorporated by reference. The '068 patent states that such compounds are useful for their antiestrogen and antiandrogen properties.

The inventor has made the surprising discovery that compounds of Formula I—known principally for their antiestrogen properties—may be used to treat androgen-independent prostate cancer; that is, that such compounds may be used to treat cancers that lack androgen receptors or otherwise do not depend on androgens for their growth. This discovery is particularly surprising in light of evidence that similar antiestrogens, most notably tamoxifen, have no effect on androgen-independent prostate cancer.

The inventor has demonstrated that prostate cancer cells express the beta isoform of estrogen receptors, and hypothesizes that the mechanism of action of the compounds of Formula I is signaling through the beta isoform of the estrogen receptor. Preferred compounds of the invention are therefore those compounds of Formula I that are effective in blocking the beta isoform of the estrogen receptor.

An especially preferred compound for use with the method of the invention is raloxifene, a benzothiopene having the formula

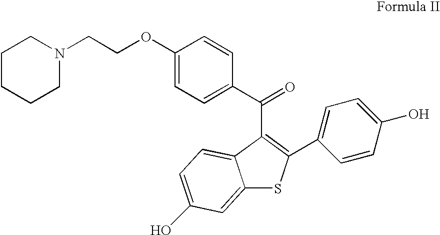

and pharmaceutically acceptable salts thereof. Raloxifene hydrochloride (raloxifene HCl) is the preferred salt of the compound of Formula II.

Another especially preferred compound for use with the method of the invention is arzoxifene, a benzothiopene having the formula

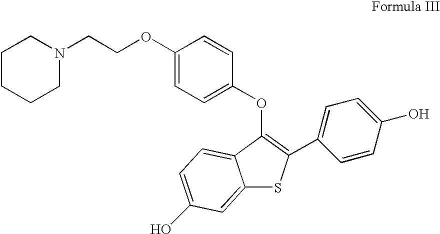

and pharmaceutically acceptable salts thereof. Arzoxifene hydrochloride (arzoxifene HCl) is the preferred salt of the compound of Formula III.

In another embodiment of the invention, one administers to a mammal a prodrug of the compound of Formula I, or, preferably, a prodrug of the compound of Formula II or Formula III.

As used herein, the term “prodrug” refers to any compound that is converted into an active benziothiopene by metabolic processes within the body. There are various reasons why one might wish to administer a prodrug of the compounds of Formula I, II, or III rather than the compound itself. Depending on the particular compound (or its salt) that one uses, a prodrug might have superior characteristics as far as solubility, absorption, stability, release, toxicity, and patient acceptability are concerned. It should be readily apparent to one of ordinary skill in the art how one can make a prodrug of any compound of the invention. There are many strategies for doing so. One can replace one or more of the oxygen atoms with hydrogen, for example. Such prodrugs are converted in vivo by enzymatic hydroxylation to active benzothiopenes. Other prodrugs should be readily apparent to one of ordinary skill in the art.

The administration of compounds of Formula I, II, or III (including their salts and prodrugs of these formulas) may be used as single agent therapy or as an adjuvant to established therapy, such as chemotherapy, surgery, or any other therapy known in the art to treat cancer.

The appropriate dosage of the compounds of the invention depends on the age and weight of the individual to be treated, whether the compound is being used as single agent or adjuvant therapy, the type of androgen-independent cancer (e.g., whether it is an adenocarcinoma, sarcoma, squamous cell carcinoma, ductal transitional carcinoma, or other prostatic cancer), the progression of the cancer (e.g., whether it has metastasized or is localized), its size, location, and other factors well known to those skilled in the art of oncology. In general, doses of between 10 mg and 300 mg per day may be used; 60 mg per day is preferred for most cases. The administration of either raloxifene HCl or arzoxifene HCl at a single dose of 60 mg per day is especially preferred.

One preferably administers compounds of the invention orally, though one can also administer them by intravenous and intramuscular injection. In an especially preferred embodiment, one administers raloxifene HCl as a solid tablet containing 60 mg raloxifene HCl and excipients. In another especially preferred embodiment, one administers arzoxifene HCl as a solid tablet containing 60 mg arzoxifene HCl and excipients.

Compounds of the invention are generally prepared by uniformly combining a compound of Formula I, II, or III with a pharmaceutically acceptable liquid carrier, a finely divided solid carrier, or both, and then shaping the product if necessary. As used herein, “pharmaceutically acceptable carrier” refers to a carrier that is compatible with the other ingredients of the formulation and does not harm the subjects to which it is administered. Suitable such carriers include, for example, water, alcohols, natural or hardened oils and waxes, calcium and sodium carbonates, calcium phosphate, kaolin, talc, and lactose.

Formulations according to the invention are preferably combined with one or more excipients such as the following: preservatives, such as ethyl-p-hydroxybenzoate; suspending agents such as methyl cellulose, tragacanth, and sodium alginate; wetting agents such as lecithin, polyoxyethylene stearate, and polyoxyethylene sorbitan mono-oleate; granulating and disintegrating agents such as starch and alginic acid; binding agents such as starch, gelatin, and acacia; lubricating agents such as magnesium stearate, stearic acid, and talc; flavoring and coloring agents, and any other excipient conventionally added to pharmaceutical formulations.

Formulations of the present invention suitable for oral administration may be presented in any of the following forms: discrete units such as capsules, cachets, or tablets each containing a predetermined amount of the active ingredient; powder or granules; solutions or suspensions in an aqueous liquid or a non-aqueous liquid; or, as oil-in-water liquid emulsions or water-in-oil emulsions, and any other form suitable for oral administration.

### EXAMPLES

The following examples are typical of the procedures that may be used to treat, in accordance with the methods of the invention, patients suffering from androgen-independent prostate cancer, or to evaluate the efficacy of compounds of the invention which may be used to treat such patients. Modifications of these examples will be readily apparent to those skilled in the art who seek to treat patients whose condition differs from those described here.

### Example I

### Treatment of Male Patients with Androgen-independent Prostate Cancer

Three male patients were selected for treatment on the basis of established diagnosis with prostate cancer. All three patients had failed standard androgen withdrawal therapy. Average prostate-specific antigen (“PSA”) levels for each patient at the beginning of treatment was about 20-30 ng/ml. Hormone ablative therapy, in some cases aggressively administered as long as 36 months, had failed, manifested by a continued rise in PSA levels in each patient. The lack of response to such therapy indicated that the prostate cancer in each patient was (or had become) androgen-independent.

Each patient received orally 60 mg of raloxifene HCl once a day for a minimum of 30 days. PSA levels in each patient declined >50% in each patient after this period, demonstrating that raloxifene HCl can produce dramatically beneficial results in patients suffering from androgen-independent prostate cancer. The longest treated patient, as of the filing of this application, is now 11 months since beginning therapy with a continued PSA<0.4 ng/ml and improvement in his bone scan.

### Example II

### Inhibition of Tumor Growth in Androgen-independent Xenograft Model

The inventor used an antigen-independent xenograft model of human prostate tumor cells (CUR22R) to raise tumors in male and female nude mice. Tumors developed over a period of 2-3 weeks into palpable masses. 10 mice were randomly assigned to a treatment group and 10 mice were assigned to a control group.

0.85 mg/kg of raloxifene HCl was administered daily by subcutaneous injection to each mouse in the treatment group. Mice in the control group received an injection of saline solution. After 30 days, tumors from surviving mice were surgically removed and visibly examined. Tumor size was reduced by 60% in mice from the treated group as compared to mice from the control group.

What is claimed is:

1. A method of treating a mammal with prostate cancer, the method comprising administering to the mammal an effective amount of a compound having the formula 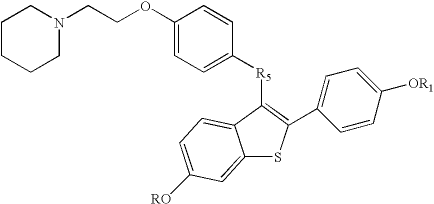
or pharmaceutically acceptable salts thereof,
wherein R and R₁ are each independently selected from the group consisting of hydrogen, —COR₂, —COR₃, and R₄;
R₂ is selected from the group consisting of hydrogen, C1-C14 alkyl, C1-C3 chloroalkyl, C1-C3 fluoroalkyl, C5-C7 cycloalkyl, C1-C4 alkoxy, and phenyl;
R₃ is phenyl with at least one substitution selected from the group consisting of C1-C4 alkyl, C1-C4 alkoxy, hydroxy, nitro, chloro, fluoro, trichloromethyl, and trifluoromethyl;
R₄ is selected from the group consisting of C1-C4 alkyl, C5-C7 cycloalkyl, and benzyl; and
R₅ is selected from the group consisting of oxygen and —C═O.

2. The method of claim 1, wherein the compound is administered in an effective amount of between about 0.1 mg and 10 mg per kg of body weight of the mammal per day.

3. The method of claim 2, wherein the compound is administered in an effective amount of between about 0.5 mg and 2 mg per kg of body weight of the mammal per day.

4. The method of claim 3, wherein the compound is administered orally.

5. The method of claim 1, wherein R and R₁ are both hydrogen.

6. The method of claim 5, wherein R₅ is oxygen.

7. The method of claim 5, wherein R₅ is —C═O.

8. A method of treating a mammal with prostate cancer, the method comprising administering to the mammal an effective amount of a prodrug of a compound of the formula 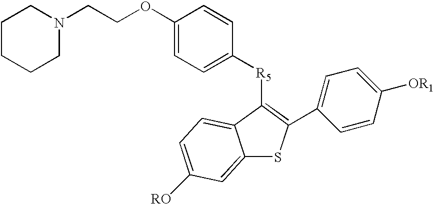
or pharmaceutically acceptable salts thereof,
wherein R and R₁ are each independently selected from the group consisting of hydrogen, —COR₂, —COR₃, and R₄;
R₂ is selected from the group consisting of hydrogen, C1-C14 alkyl, C1-C3 chloroalkyl, C1-C3 fluoroalkyl, C5-C7 cycloalkyl, C1-C4 alkoxy, and phenyl;
R₃ is phenyl with at least one substitution selected from the group consisting of C1-C4 alkyl, C1-C4 alkoxy, hydroxy, nitro, chloro, fluoro, trichloromethyl, and trifluoromethyl;
R₄ is selected from the group consisting of C1-C4 alkyl, C5-C7 cycloalkyl, and benzyl; and
R₅ is selected from the group consisting of oxygen and —C═O.

9. The method of claim 8, wherein the compound is administered in an effective amount of between about 0.1 mg and 10 mg per kg of body weight of the mammal per day.

10. The method of claim 9, wherein the compound is administered in an effective amount of between about 0.5 mg and 2 mg per kg of body weight of the mammal per day.

11. The method of claim 10, wherein the compound is administered orally.

12. The method of claim 8, wherein R and R₁ are both hydrogen.

13. The method of claim 12, wherein R₅ is oxygen.

14. The method of claim 12, wherein R₅ is —C═O.

15. A method of treating a mammal with prostate cancer, the method comprising administering to the mammal an effective amount of a compound having the formula 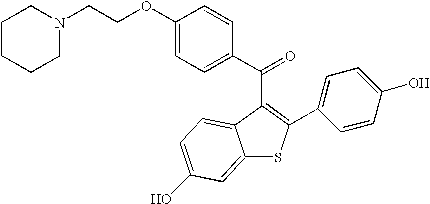
or pharmaceutically acceptable salts thereof.

16. The method of claim 15, wherein the compound is administered in an effective amount of between about 0.5 mg and 2 mg per kg of body weight of the mammal per day.

17. A method of treating a mammal with prostate cancer, the method comprising administering to the mammal an effective amount of a prodrug of a compound of the formula 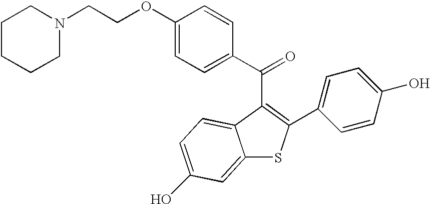
or pharmaceutically acceptable salts thereof.

18. The method of claim 17, wherein the compound is administered in an effective amount of between about 0.5 mg and 2 mg per kg of body weight of the mammal per day.

19. A method of treating a mammal with prostate cancer, the method comprising administering to the mammal an effective amount of a compound having the formula 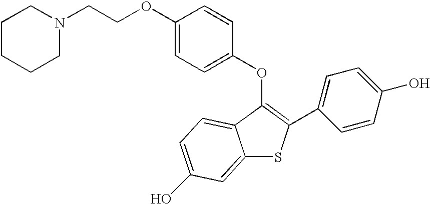
or pharmaceutically acceptable salts thereof.

20. The method of claim 19, wherein the compound is administered in an effective amount of between about 0.5 mg and 2 mg per kg of body weight of the mammal per day.

21. A method of treating a mammal with prostate cancer, the method comprising administering to the mammal an effective amount of a prodrug of a compound of the formula 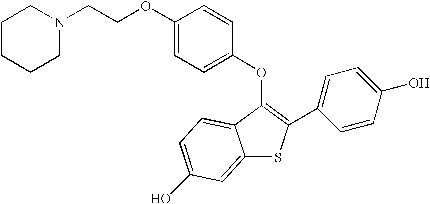
or pharmaceutically acceptable salts thereof.

22. The method of claim 21, wherein the compound is administered in an effective amount of between about 0.5 mg and 2 mg per kg of body weight of the mammal per day.
